(19) United States
(12) Patent Application Publication
(10) Pub. No.: US 2024/0016376 A1
(43) Pub. Date: Jan. 18, 2024

(54) Surgical Clip for Endoscopic Carpal Tunnel Surgery and Other Procedures

(71) Applicant: IntraVu, Inc., Redwood City, CA (US)

(72) Inventors: Theofilos Kotseroglou, Hillsborough, CA (US); Stephanos Papademetriou, Woodside, CA (US); Ulrich R. Haug, Campbell, CA (US); Richard Jeremy Hadden, White Salmon, WA (US)

(21) Appl. No.: 17/867,408

(22) Filed: Jul. 18, 2022

(51) Int. Cl. A61B 1/32; A61B 1/317; A61B 1/00; A61B 1/06; A61B 17/32; A61B 1/04

(52) U.S. Cl. CPC A61B 1/32; A61B 1/317; A61B 1/00154; A61B 1/0684; A61B 17/320016; A61B 1/04; A61B 1/00009; A61B 1/00087

(57) ABSTRACT

A clip for use in carpal tunnel surgery and similar surgeries has two separate clip sections, each with a proximal end that snaps together with the opposing proximal end of the other section, to make an assembled clip with a space between the sections except at proximal ends. At least one of the clip sections can be configured as a dilator, with a tapered distal end and without sharp edges. The two clip sections form endoscope and live channels to receive a viewing endoscope and a knife, inserted through the proximal end of the assembled clip device. Illumination is provided, either within the clip or from the scope of another inserted device, to enable viewing through the endoscope during a procedure. An embodiment of the clip device is formed with a rotation hub, such that the two clip sections can be swung apart or used together in a surgical procedure.

### BACKGROUND OF THE INVENTION

This invention concerns endoscopic surgery, and is particularly directed to a surgical clip for carpal tunnel surgery and similar surgeries, providing a guide for a cutting knife and for a viewing scope.

In 1992, the Agee technique for the release of the transverse carpal ligament endoscopically was established, and now it is widely accepted as an alternative to open surgical procedure. Further, recognition of specific anatomic landmarks and varied patients' anatomy helps to avoid complications and improves speed and outcomes of Endoscopic Carpal Tunnel Release (ECTR).

Surgical clips or guides for carpal tunnel surgery and similar procedures are known. See, for example, U.S. Pat. Nos. 10,245,062, 10,806,481, 11,000,303 and 11,033,291. Ease of use and convenience in the surgical procedure are issues with the clip devices. The clips are formed as a pair of elongated prongs or sections with a gap between them, insertable through a surgical slit to engage the upper and lower surfaces of a particular tissue to be cut, such as the transverse carpal ligament. This ligament can be the source of what is called carpal tunnel syndrome, a painful condition resulting from repetitive use of the hand in particular ways. The tunnel refers to the space under the transverse carpal ligament, in which the median nerve can be compressed, causing the pain.

The pain of carpal tunnel syndrome typically can be alleviated by surgery such as the Agee technique in which the patient's transverse carpal ligament is severed. This can be done via a small cut in the wrist area, for entry of a surgical clip as described above. A surgical knife is inserted into a channel of the surgical clip, enabling the cutting of the ligament. The clip serves as a guide for the knife and for a viewing scope and protects the surrounding tissues.

Some of the prior surgical clips have had the upper and lower sections integrally formed as a single unit; others have been separable, with attachment required prior to insertion.

The invention provides an efficient surgical clip device with multiple functions and efficiency of use.

### SUMMARY OF THE INVENTION

The invention provides an improved surgical clip, particularly for carpal tunnel surgery procedures. In one form the clip comprises two separate clip sections, an upper and a lower section. Each has a proximal end with a snap-together device for connecting the two to provide an assembled clip with the proximal ends of the sections firmly together and defining a space between the two sections except at the proximal ends. A first of the clip sections functions as a dilator, having a tapered distal end for insertion between the transverse carpal ligament and tissue immediately above. A first step in the procedure with this embodiment is insertion of the dilator clip section, alone without the other section. Once this has been accomplished, a second of the two clip sections is inserted below the carpal ligament, and this is snapped together with the dilator section to form the assembled clip in place for the surgery.

One or both of the clip sections has channels configured to receive a viewing endoscope and a knife. Access openings can be provided in one or both proximal ends of the clip sections for insertion of these implements in support of the surgery.

Illumination can be provided either as a fixed feature of one of the sections, e.g. one or more LEDs, or illumination can be integral with the scope or with the knife to provide illumination when inserted.

In a variation, the endoscope can be a permanent component of one section of the clip, and it may be slidable within a channel, or fixed in position with a large field of view.

In a preferred embodiment the connection of the two clips is accomplished during the last few millimeters of movement of the lower clip section, whereby the proximal ends are secured together by sliding motion.

In another preferred embodiment both the upper and lower clip sections are identical, each having a tapered end for use as a dilator. This allows the off-center channel which receives the knife to be used for either a left or right hand operation.

In another variation more than one endoscope is permanently included in one or both of the clips. Images from two scopes can be integrated on a computer display, thus to present a view of the entire channel without sliding a scope.

In a further variation a surgical knife can be integrated into one of the clip sections, slidable into a channel but not removable. A specific form of such a knife is a rotatable knife blade with a thumb dial on the exterior of the clip, so as to manipulate the angle of the knife blade and enable greater precision in the surgery.

The surgical clip of the invention can be used in surgeries other than carpal tunnel release, such as cubital tunnel release in the elbow.

### DESCRIPTION OF THE DRAWINGS

FIG. 1 is an exploded view in perspective showing an embodiment of the invention, with upper and lower arms or clip sections of a carpal tunnel guide clip.

FIG. 2 is a perspective view showing the clip as assembled.

FIG. 3 is a perspective view of a clip showing an endoscope used with the clip.

FIG. 4 is a schematic view showing wrist anatomy including the transverse carpal ligament.

FIG. 5 is another perspective view showing a clip with an endoscope and a knife.

FIG. 6 is a similar view at a different angle.

FIGS. 7, 8 and 9 are views showing the clip with an endoscope and knife.

FIGS. 10 and 11 are perspective views showing the clip with a knife, with one arm or section of the clip removed for clarity.

FIG. 12 is a perspective view showing a different form of surgical clip of the invention.

FIGS. 13A, 13B and 13C show another version of the clip wherein the clip legs or arms open/closed about a transverse axis.

FIGS. 13D, 13E, 13F, 13G, 13H and 131 are perspective views showing components and details of the clip embodiment shown in FIGS. 13A to 13C.

FIG. 13J is a sectional view showing the knife as positioned in the clip.

FIGS. 14 and 15 are perspective views showing a pick and a knife that can be used with the invention.

FIG. 16 is an exploded perspective view showing an implement inserted in the clip of the invention, and a knife that can be inserted when the implement is removed.

FIGS. 17 and 18 are perspective and plan views showing a set of parts that can be packaged together for use of a surgical clip of the invention.

FIG. 19 is a perspective view showing a scope with two shafts used with a clip of the invention, wherein one shaft can be for illumination.

FIG. 20 is a view showing the same clip as in FIG. 19, with a cross section cut through the two shafts of the scope.

FIGS. 21A and 21B are perspective views showing a version of the clip of the invention used with a particular scope.

### DESCRIPTION OF PREFERRED EMBODIMENTS

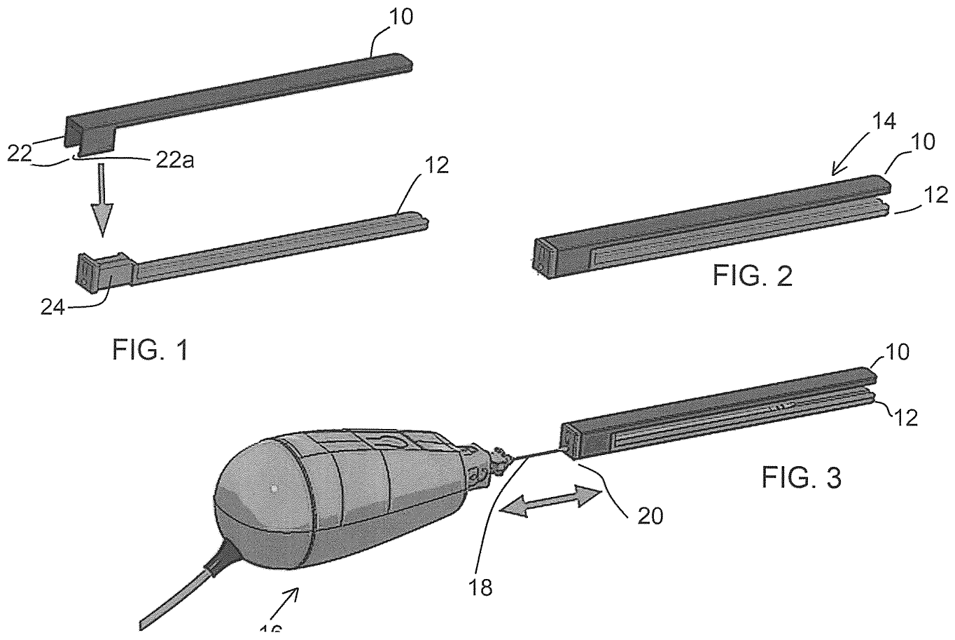

FIGS. 1, 2 and 3 show an embodiment of the invention in which two arms or legs 10 and 12 of a guide clip are snapped together to form an assembled guide clip 14 as shown in FIG. 2. In FIG. 3 an endoscope 16 with a narrow shaft 18 is inserted into a leg of the clip, which in this example the lower leg 12, for which a hole is provided at the proximal end 20 of the assembled clip. This opening and channel for the scope shaft 18 can be on either the upper or lower leg 10 or 12. The invention of FIG. 1 can take various forms, in this case being a pair of gripping arms 22 on the upper leg 10, snapping closely over a rectangular body 24 at the proximal end of the lower leg, as shown. The gripping arms 22 preferably have a lip 22a at the bottom end of each, to make a more secure and permanent assembly. The positions of the structures 22 and 24 can be reversed.

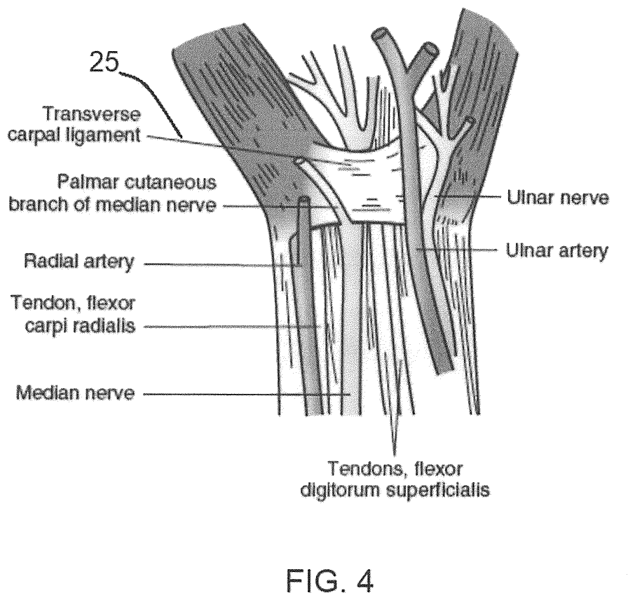

FIG. 4 shows the transverse carpal ligament 25 in the wrist anatomy. The clip of the invention is engaged with arms under and over the ligament; the drawing shows proximity of veins and nerves.

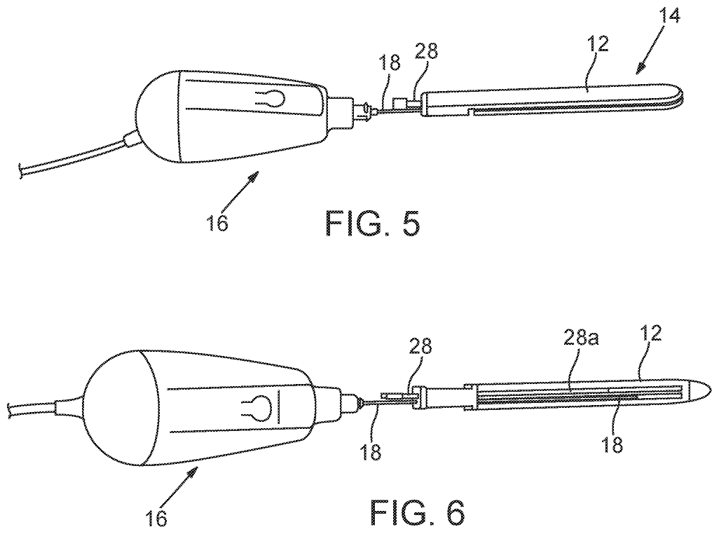

FIGS. 5 and 6 show the exemplary scope 16 with its elongated scope shaft 18 extended into one of the legs of the assembled clip 14. Adjacent to the needle 18 is a cutting knife 28, the proximal end of which is seen in FIG. 5, and the blade 28a of which can also be seen in the slightly rotated view of FIG. 6. FIG. 6 is shown with one of the clip arms removed, that being the upper arm 10 in the embodiment shown in FIG. 1, revealing the lower arm 12 in the drawing, to indicate the scope shaft 18 and the knife blade 28a as positioned in the clip.

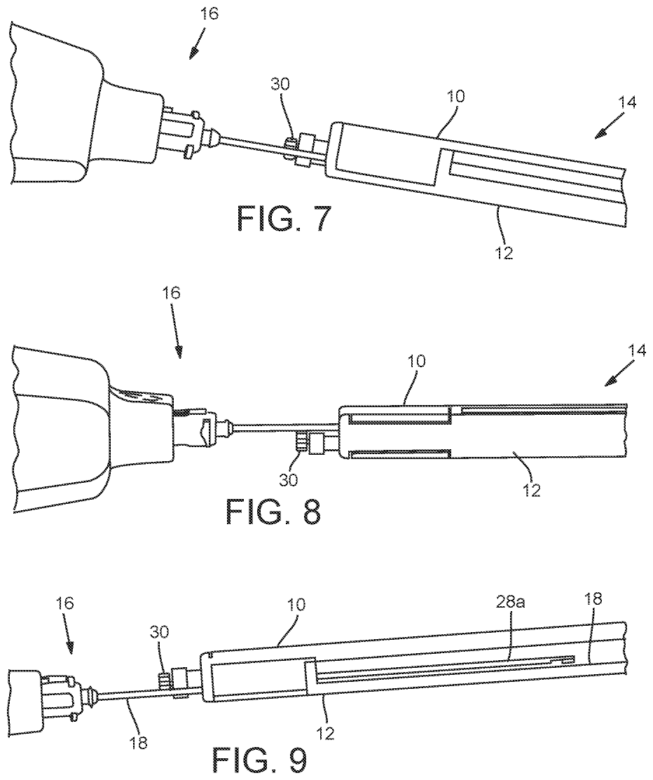

FIGS. 7, 8 and 9 are enlarged views showing the clip and scope. FIGS. 7 and 8 show the assembled clip 14, while FIG. 9 shows the lower clip 12, the upper clip having been removed. This example shows a different form of cutting knife. The knife is rotatable about a long axis, and has a rotating knob or thumb wheel 30 protruding out the proximal end of the clip. This enables the surgeon to rotate the angle of the cutting blade for greater precision cutting a specific area of the carpal ligament or other tissue. FIG. 9 reveals the scope shaft 18 as it is positioned in a channel of the clip, and the knife blade 28a, the channel for which is formed by both upper and lower clip legs when assembled.

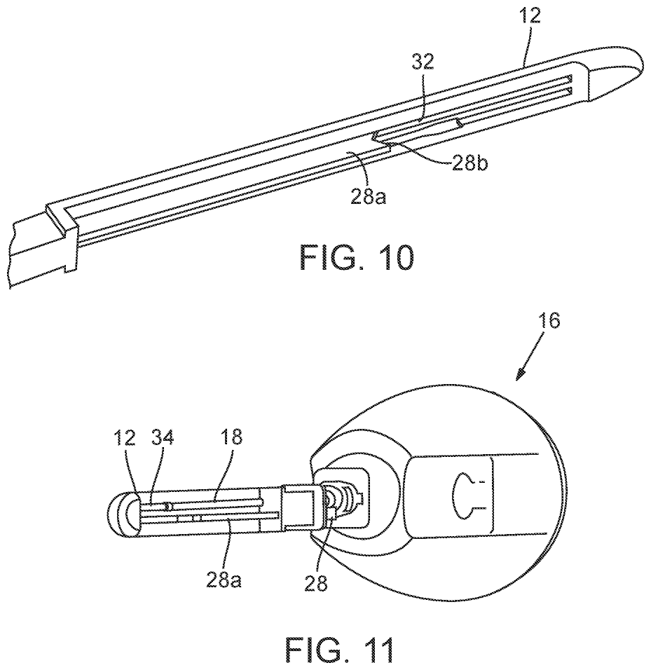

FIGS. 10 and 11 are further perspective views indicating the use of a cutting knife in the surgical clip device of the invention. In both views the lower section or leg 12 of the clip is shown, with the upper leg 10 removed for clarity. In this embodiment the knife is not rotatable. The knife blade 28a and its cutting end 28b, which can be V-shaped as shown, are indicated residing in a slot 32, the lower leg part of which is shown in these drawings. The opposing clip leg will have a facing slot to complete the knife slot. In FIG. 11 the endoscope 16 is also shown with its shaft 18 extending into the channel 34 of the clip, the lower leg 12 of which is shown.

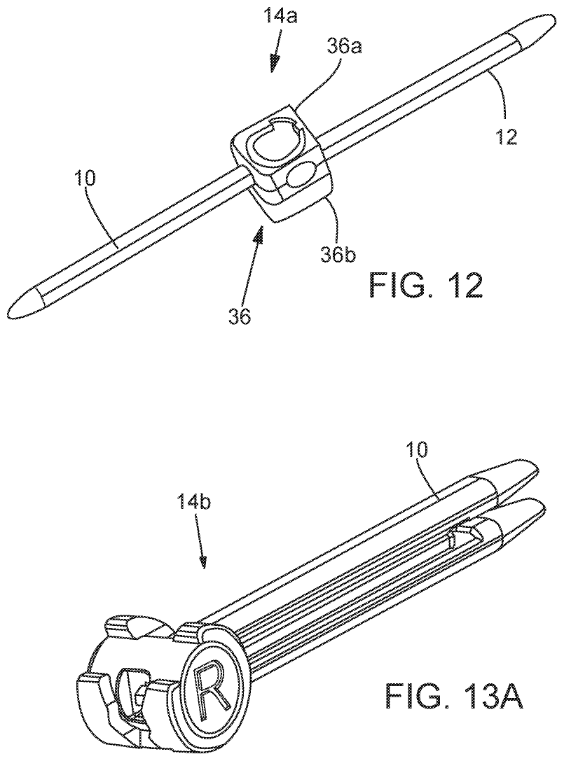

FIG. 12 shows a variation of the invention, in which a surgical clip 14a has upper and lower legs 10 and 12 which swivel relative to one another on what can be considered a vertical axis, i.e. both swinging in a horizontal plane. That is, the axis is perpendicular to a plane which would contain either of the legs. A rotational connection hub 36 provides for the swiveling, being composed of two snap-together hub portions 36a and 36b. With this configuration of a surgical clip, the legs can be swung apart to a linear “open” configuration, in 180° position as shown, in which one or the other of the legs 10 or 12 is used as an obturator or dilator. Then, once the tissues have been separated as needed, the device can be pulled back and the legs swiveled to the clip-forming position, i.e. 0°, not shown in this view.

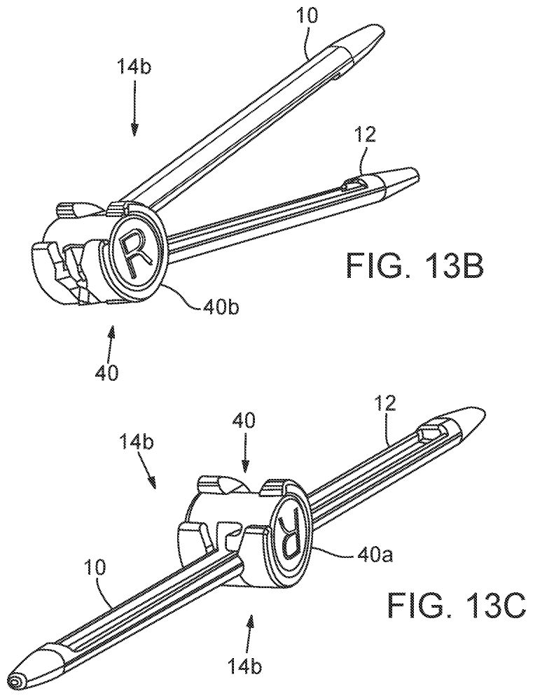

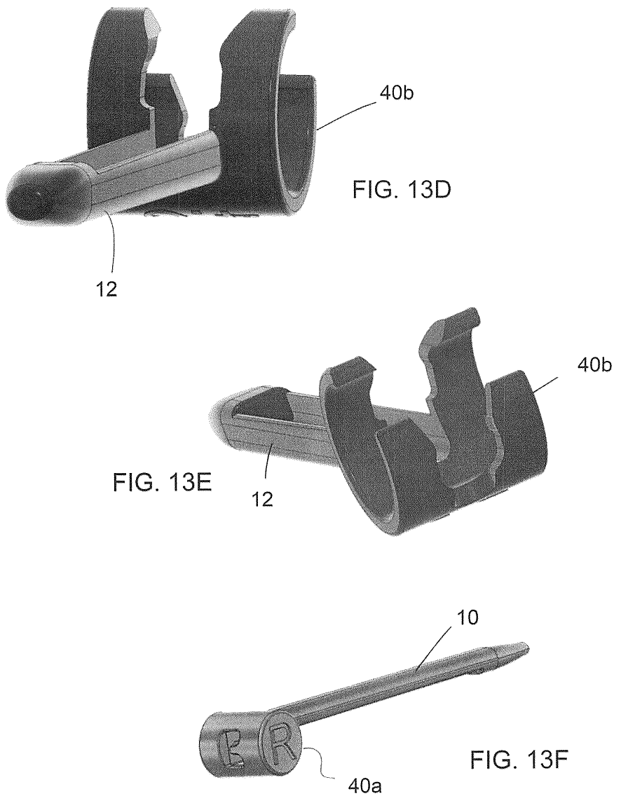

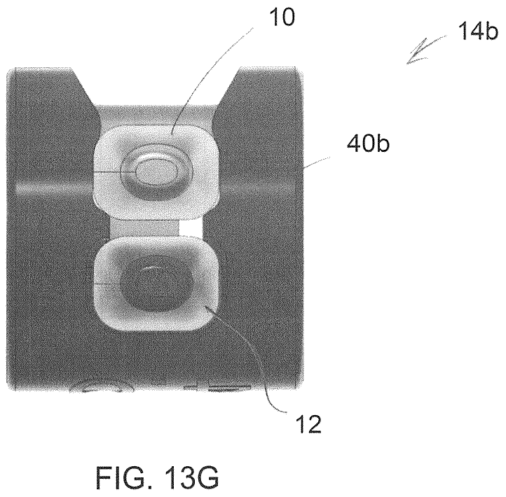

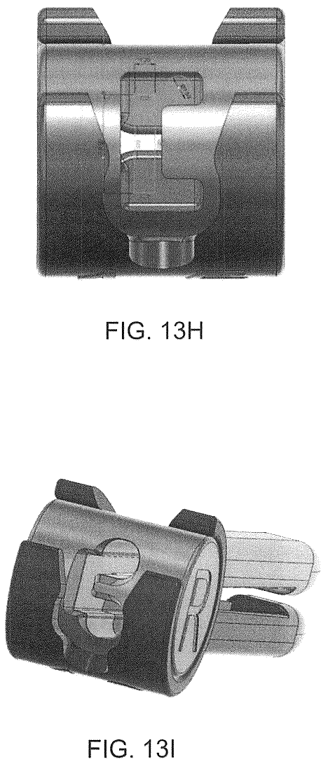

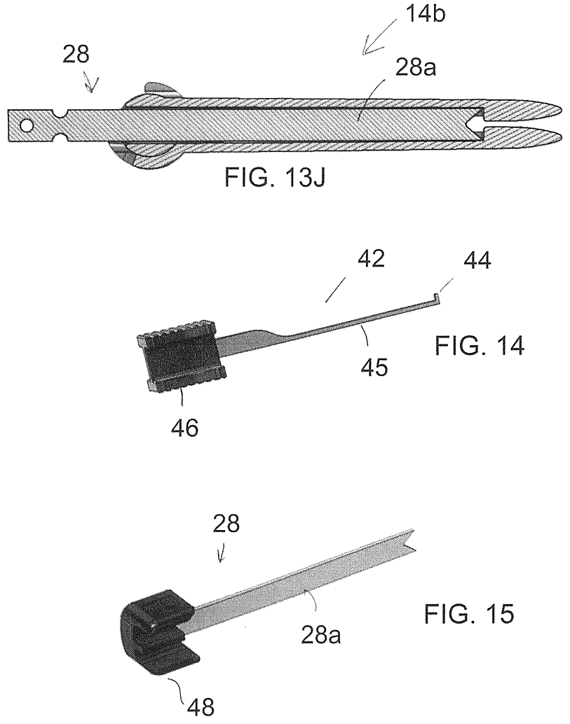

FIGS. 13A through 13J show another version of a surgical clip 14b, with clip legs relatively rotatable on a lateral axis, i.e. an axis essentially parallel to a plane in which one of the legs lies and perpendicular to the length of the leg. The clip device can be used, as shown in FIG. 13C, in a 180° open position in which one leg can be used as an obturator. FIG. 13B shows the clip in a position between that of 13A and that of 13C.

The two clip legs 10 and 12 can be in a barrel type swivel clip connection as indicated at 40. This barrel type swivel is separable, so that the barrel 40a, which is a part of the upper clip leg 10, can be snapped into a cylindrical receiver hub 40b, the hub being configured to allow movement of the upper clip leg as indicated in the drawings, between the 0° clip configuration of FIG. 13A (procedure mode) and the 180° position shown in FIG. 13C (dilator mode). The swivel connection imposes high friction so as to hold a selected angular position. The separability of the clip sections can be only for the purpose of initial assembly.

FIGS. 13D and 13E further show the cylindrical hub 40b on the lower leg 12, while FIG. 13F shows the barrel 40a on the upper leg 10. FIG. 13G shows the surgical clip device 14b in the procedure mode, looking from the clip tip end back toward the hub. FIG. 13H is a view from the opposite side showing the two channels, closed at the distal ends. In FIG. 13I a variation is shown in which the clip openings and channels are closer together, allowing for a smaller clip hub.

FIG. 13J is a cross-section showing limitation of knife movement into the channel.

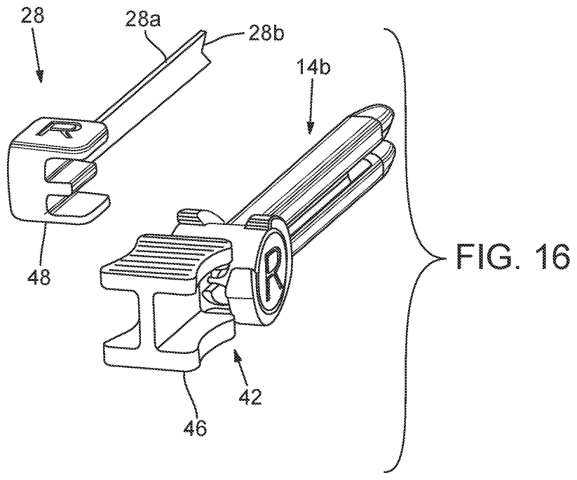

FIGS. 14 and 15, as well as FIG. 16, show two tools that can be inserted into a channel or slot of the surgical clip. In FIG. 14 is a measurement pick 42 that has a hook 44 on its distal end. A scale preferably is included on the shaft 45 for measurement of the ligament. FIG. 15 shows a knife 28 such as shown in other views and discussed above. In both tools 42 and 28 a handle is shown at the proximal end of the tool, as at 46 and 48, respectively.

In FIG. 16 the clip 14b is shown in the procedure configuration, with a tool such as the measurement pick 42 inserted into the clip. The handle 46 extends out distally. The cutting knife 28 is also shown in FIG. 16, for insertion into the clip at the appropriate time.

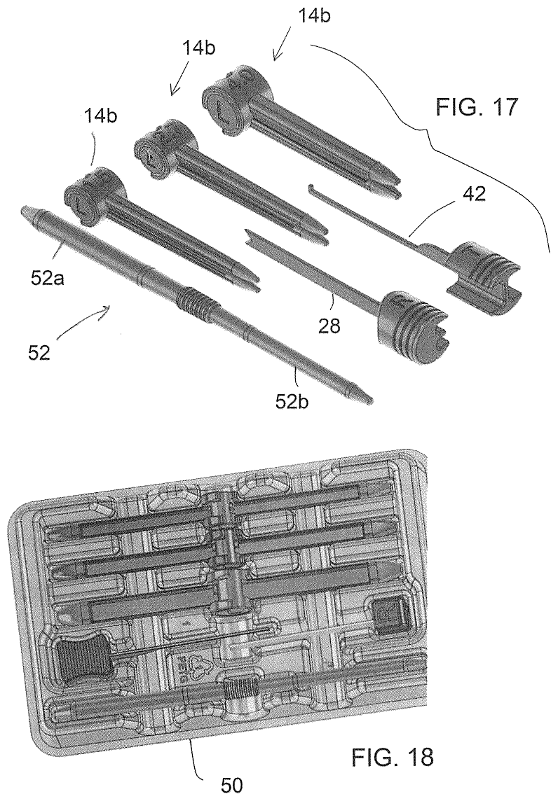

FIGS. 17 and 18 show a kit of carpal tunnel surgical tools, which may be packaged in an instrument tray 50 that includes three different surgical clips 14b of the configuration described above including provision for swiveling about a horizontal axis. The tissue measurement pick 42 and the knife 28 are also included. Another implement that can be a part of the set contained in the tray 50 is a dilator tool 52 with ends 52a and 52b of different sizes for performing a dilating function. This tool is optional, as the swiveling clip 14b itself includes the two legs each of which can be used as a dilator.

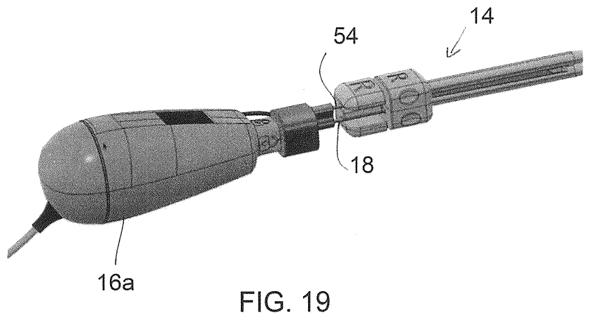

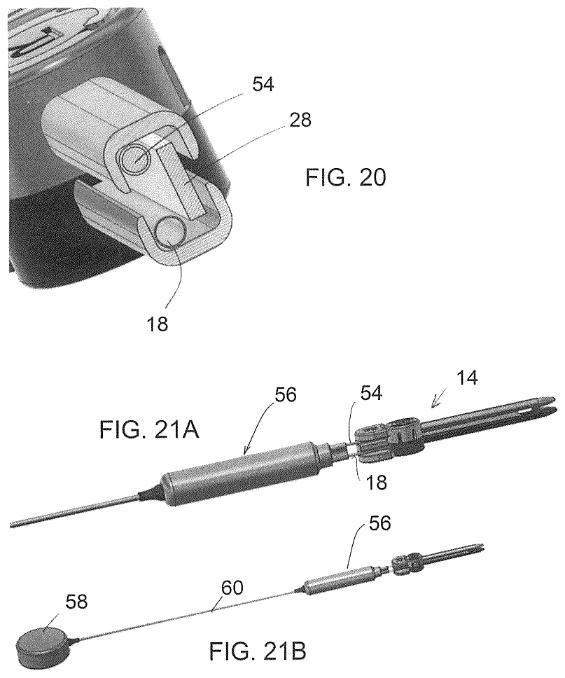

FIGS. 19, 20 and 21 show another embodiment, this one specific to the use of a scope 16a having two parallel shafts 18 and 54. The shaft 18 is the endoscope shaft, for viewing the tissue and use of the surgical clip 14 during a procedure. The second shaft 54 is a separate illumination shaft, i.e. the shaft 54 can carry an optical fiber bundle. In this case, in which the illumination is separated from the scope camera, the instrument can use a somewhat larger camera, which is less costly and can be a disposable item. When the scope and an illumination are carried in the same shaft, the camera and optics must be smaller and therefore of higher cost. In use of the scope 16a in the case of separated camera and illumination, the illumination is made from above or below, as the case may be, while the images are generated from the other shaft 18.

In another form of the device shown in FIG. 19, both shafts can carry both illumination and imaging capability, thus both shafts comprise scopes on their own. This provides more information for the surgeon, and the two generated images can be stitched together by software into one view if desired.

FIGS. 21A and 21B schematically indicate the two shafts 18 and 54 in cross section, along with the knife 28, all guided in a channel or channels formed in the surgical clip. FIGS. 21A and 21B show a different style of scope 56, having the two shafts 18 and 54 as discussed above. FIG. 21A shows an enlarged portion of FIG. 21B. In this embodiment the modified scope 56 is connected to a wi-fi or other wireless transmitter 58, by a wire or shaft 60, to send images to a computer and display wirelessly. This can be an inexpensive, disposable scope assembly. Although the drawing indicates the snap-together clip of the first-described embodiment, this can be any embodiment of the clip. Once the clip is closed and inserted and has captured the ligament between the legs, it can accept a dual scope into the top and the bottom channels as shown.

### Procedure for Use of the Apparatus of the Invention

### Marking Anatomic Landmarks

Mark key anatomical landmarks on the patient's hand using a sterile skin marking pen: the estimated position of the Hook of the hamate, 3^{rd} webbing space (for transverse carpal ligament) and the proximal wrist crease (for incision line).
Making the Incision along Wrist Flexion Crease Choose a line about 1 cm proximal to the pronounced Wrist Flexion Crease. Make a 1-2 cm transverse incision between the tendons of the flexor carpi radialis and the flexor carpi ulnaris.
CAUTION: If the palmar cutaneous branch of the median nerve is visualized in the radial side of the wound, it shall be gently isolated and retracted.
Optional: In challenging anatomy, elevating the distal edge of the fascia with double-pronged skin hooks from the underlying flexor tenosynovium (ulnar bursa) allows opening a plane between the synovium and the deep side of the transverse carpal ligament. Use tenotomy scissors to gently spread and separate the forearm fascia from the underlying tenosynovium of the ulnar bursae.

### Carpal Tunnel Dilation

Insert small end of dilator into carpal tunnel—aim for space between 3^{rd} and 4^{th} metacarpal bones (could be identified by palpating with non-instrument hand). Employ twisting and turning motion in order to advance dilator safely. Repeat with wide-end of dilator to further dilate the tunnel.

### Creating Subcutaneous Channel

Repeat the dilation step to create a subcutaneous channel; make sure to use same anatomical landmarks to align with the carpal tunnel made in the dilation step.
Optional: CarpalVu clip of next-to-intended procedure size can be used instead of dilator. Note tips of clip (these preferably are symmetrical on both ends) are designed to combine function of dilator and synovium elevator that allows performing dilation and tissue separation at the same time. The roughness of the transverse fibers of the transverse carpal ligament (ribbed effect²) can be felt by pressing clip tip against ligament while advancing tip distally. This is an indication of ligament surface is clean and ready for a cut.

### Inserting CarpalVu Clip

Fold CarpalVu clip in half. Use fresh clip of appropriate size (1.5 mm, 2.7 mm, and 4.0 mm) matching videoscope size; clean internal surface of CarpalVu clip alleviates lens fogging problem. Make sure the following elements are addressed:
R/L markings—for right hand surgery (R) is facing thumb; for left hand—(L);
One end of CarpalVu clip is inserted in carpal tunnel (ref. step VII);
The other end of CarpalVu clip is inserted into subcutaneous channel (ref. step VIII);
The knife slot on the proximal side of the clip shall be ulnar to scope ports.

### Inspection/Visualization of Ligament

Use available scope to inspect transverse carpal ligament's superficial (top port) and sub-synovial (bottom port) surfaces. Surfaces shall be clean from remaining synovium and other debris. Advance CarpalVu clip distally if ligament's distal edge is not encased by clip.
CAUTION: Consider alternative options if there no clear identification for the transverse carpal ligament's distal edge (typically characterized by a transition from ligament to fat) or edge is not encased by clip.
Optional: use supplied measuring pick to confirm location of ligament's distal edge. Measuring pick can also be used to remove remaining synovium.

### Release/Cut of Ligament

Insert and slide antegrade blade distally to complete transection; only single pass is required. CarpalVu clip is designed to securely grasp the ligament and stop the knife advancing beyond distal end of clip.
Optional: scope can be left inserted to observe knife advancement either in in the carpal tunnel or subcutaneous channel.

### Removing CarpalVu Clip

Once transection is made, remove the knife. Inspect transverse carpal ligament being released; cut edges of ligament shall be clean and with no intact fiber remaining. Remove videoscope. Note that soft fat pad tissue right next to distal end of ligament may remain intact; complete release is indicated by the observed retraction of the two sections of the ligament in radial and ulnar directions. Carefully pull out clip in motion that aligns with direction of insertion.
Close and dress the wound.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

What is claimed is:

1. A clip for performing a carpal tunnel surgery procedure, comprising:
two separate clip sections, each with a proximal end with means to manually snap together the proximal ends of the two sections to make an assembled clip with a space between the sections except at the proximal ends,
a first of the two clip sections configured as a dilator with a tapered distal end and minimal thickness at a distal tip of the distal end and without sharp edges at the distal tip,
a second of the two clip sections having scope and knife channels configured to receive a viewing endoscope and a knife, with access openings at the proximal ends of the second section for insertion of the endoscope and the knife to extend essentially through the length of the sections,
the first of the clip sections also having a knife channel positioned to be juxtaposed with the knife channel of the second clip section, so that the knife channels of the two sections when assembled will accommodate a knife, and
illumination means for lighting the space between sections so as to enable viewing through the endoscope,
whereby the first section can be used as a dilator, separated from the second section and inserted between a carpal ligament of a patient and tissue above the ligament to separate the tissue from the ligament, after which the second section can be inserted below the ligament essentially parallel to the dilator and then snapped together with the dilator via said proximal ends to make the assembled clip, then the endoscope and the knife can be inserted through the channels with illumination, and the ligament can be cut with the knife.

2. The clip of claim 1, wherein the illumination means comprises an LED fixed within a channel of one of the clip sections.

3. The clip of claim 1, in combination with said knife, the knife including said illumination means.

4. The clip of claim 1, wherein the means to manually snap together makes a permanent connection.

5. The clip of claim 1, wherein the endoscope is integrally contained in one of the clip sections.

6. The clip of claim 5, wherein the endoscope is slidable within a channel of the clip section.

7. The clip of claim 5, wherein the endoscope is fixed in position in a channel of the clip section, the endoscope having a wide angle field of view.

8. The clip of claim 1, including the endoscope included in a channel of the second clip section in a position to view the attachment of the lower section to the upper section.

9. The clip of claim 1, wherein the means to manually snap together the proximal ends comprises a slide and lock connection requiring the two sections to be in contact and subjected to relative sliding to make the connection.

10. The clip of claim 1, wherein both the first and second clip sections are configured as a dilator with a tapered distal end.

11. The clip of claim 10, wherein both the first and second sections are identical, providing for use on either a left or right arm of a patient.

12. The clip of claim 1, including a dual endoscope.

13. The clip of claim 1, including a plurality of fixed endoscopes included in channels of the clips, for wider viewing, and including computer means for integrating images from the multiple endoscopes to provide a single video image.

14. The clip of claim 1, wherein the endoscope comprises a simple scope, comprising only a chip.

15. The clip of claim 1, wherein the knife is permanently contained in a channel of one of the clip sections.

16. The clip of claim 14, wherein the knife is rotatable about a longitudinal axis of the knife, including a thumb dial at a proximal end of the knife, accessible from exterior of the clip, to enable precision cutting with the knife.

17. A clip for performing a carpal tunnel or similar surgery procedure, comprising:
two separate clip sections, each with a proximal end with means to manually snap together the proximal ends of the two sections to make an assembled clip with a space between the sections except at the proximal ends,
the two clip sections having tapered distal ends and minimal thickness at a distal tip of the distal end,
a rotational hub formed by proximal ends of the two clip sections when assembled together, providing for swinging rotation of the clip sections relative to one another about a rotation axis perpendicular to the lengths of the clip sections,
the two clip sections having scope and knife channels configured to receive a viewing endoscope and a cutting knife, with access openings at the rotational hub of the assembled clip sections for insertion of the endoscope and the knife to extend essentially through the length of the clip sections, and
illumination means for lighting the space between clip sections so as to enable viewing through the endoscope,
whereby the assembled clip can be inserted through a surgical opening in a carpal tunnel surgery, enveloping the transverse carpal ligament of the patient, one clip section extending below the ligament and the other clip section separating tissue above the ligament from the ligament, then the endoscope and the knife can be advanced through the channels with illumination, and the ligament can be cut with the knife.

18. The clip of claim 17, wherein the illumination means comprises an LED fixed within a channel of one of the clip sections.

19. The clip of claim 17, wherein the endoscope is integrally contained in one of the clip sections.

20. The clip of claim 17, including a plurality of fixed endoscopes included in channels of the clips, for wider viewing, and including computer means for integrating images from the multiple endoscopes to provide a single video image.
